(19) United States
(12) Patent Application Publication
(10) Pub. No.: US 2016/0040128 A1
(43) Pub. Date: Feb. 11, 2016

(54) METHOD

(71) Applicant: PCI BIOTECH AS, Lysaker (NO)

(72) Inventors: Pål JOHANSEN, Winterthur (CH); Anders HØGSET, Oslo (NO)

(73) Assignee: PCI BIOTECH AS, Lysaker (NO)

(21) Appl. No.: 14/776,263

(22) PCT Filed: Mar. 15, 2013
(86) PCT No.: PCT/EP2013/055493

§ 371 Date: Sep. 14, 2015

(51) Int. Cl. C12N 5/0784; A61K 39/00

(52) U.S. Cl. CPC C12N 5/0639; A61K 39/0005; A61K 39/0011; A61K 2039/5154; A61K 2039/57; C12N 2529/10; C12N 2501/06; C12N 2501/999

(57) ABSTRACT

The present invention provides an in vitro method of expressing an antigenic molecule or a part thereof on the surface of a dendritic cell using a PCI method with TPCS₂ₐ at a concentration of 0.020-0.1 μg/ml, using light of a wavelength of between 400 and 500 nm. Methods of treatment such as vaccination comprising this method, together with compositions comprising said cells and uses involving said cells expressing antigenic molecules are also provided.

The present invention relates to a method of generating antigen presenting cells in vitro which may be used to generate an immune response, e.g. for vaccination, which involves using photochemical internalisation (PCI) to introduce antigenic molecules, e.g. vaccine components, into cells to achieve antigen presentation, and to antigenic, e.g. vaccine compositions, useful in such a method. The invention also provides use of cells generated by such in vitro methods for administration to a patient in vivo to elicit an immune response, e.g. to achieve vaccination.

PCI is a technique which uses a photosensitizing agent, in combination with an irradiation step to activate that agent, and is known to achieve release of molecules co-administered to the cell into the cell's cytosol. This technique allows molecules that are taken up by the cell into organelles, such as endosomes, to be released from these organelles into the cytosol, following irradiation. PCI provides a mechanism for introducing otherwise membrane-impermeable (or poorly permeable) molecules into the cytosol of a cell in a manner which does not result in widespread cell destruction or cell death.

The basic method of photochemical internalisation (PCI), is described in WO 96/07432 and WO 00/54802, which are incorporated herein by reference. In such methods, the molecule to be internalised (which for use according to the present invention would be the antigenic molecule), and a photosensitizing agent are brought into contact with a cell. The photosensitizing agent and the molecule to be internalised are taken up into a cellular membrane-bound subcompartment within the cell, i.e. they are endocytosed into an intracellular vesicle (e.g. a lysosome or endosome). On exposure of the cell to light of the appropriate wavelength, the photosensitizing agent is activated which directly or indirectly generates reactive species which disrupt the intracellular vesicle's membranes. This allows the internalized molecule to be released into the cytosol.

It was found that in such a method the functionality or the viability of the majority of the cells was not deleteriously affected. Thus, the utility of such a method, termed “photochemical internalisation” was proposed for transporting a variety of different molecules, including therapeutic agents, into the cytosol i.e. into the interior of a cell.

WO 00/54802 utilises such a general method to present or express transfer molecules on a cell surface. Thus, following transport and release of a molecule into the cell cytosol, it may be transported to the surface of the cell where it may be presented on the outside of the cell i.e. on the cell surface. Such a method has particular utility in the field of vaccination, where vaccine components i.e. antigens or immunogens, may be introduced to a cell for presentation on the surface of that cell, in order to induce, facilitate or augment an immune response.

These methods use the photochemical effect as a mechanism for introducing otherwise membrane-impermeable molecules into the cytosol of a cell in a manner which does not result in widespread cell destruction or cell death, unlike photodynamic therapy (PDT) methods which generate higher levels of reactive species to achieve cell death.

A range of photosensitizing agents are known, including notably the psoralens, the porphyrins, the chlorins and the phthalocyanins.

Photosensitizing drugs may exert their effects by a variety of mechanisms, directly or indirectly. Thus for example, certain photosensitisers become directly toxic when activated by light, whereas others act to generate reactive species, e.g. oxidising agents such as singlet oxygen or oxygen-derived free radicals, which are extremely destructive to cellular material and biomolecules such as lipids, proteins and nucleic acids.

Porphyrin photosensitisers act indirectly by generation of reactive oxygen species. TPCS₂ₐ (Disulfonated tetraphenyl chlorin, e.g. Amphinex®) has been advocated for use as a photosensitizing agent in various methods (WO03/020309), but has not been advocated for use on dendritic cells under the conditions described herein.

There remains a need for improved methods of PCI in which antigens are effectively expressed on cell surfaces. The present invention addresses this need.

The present inventors have surprisingly found that, advantageously, low doses or concentrations, i.e. in the range of 0.020-0.1 μg/ml, of the photosensitizing agent TPCS₂ₐ, in combination with particular wavelengths of light, i.e. in the blue light range of 400-500 nm, can be utilised in a method for expressing an antigen on the surface of a dendritic cell. Cells produced in this way exhibit good presentation and little apoptosis and may be used for vaccination.

Since most vaccines are taken up by antigen presenting cells through endocytosis and transported via endosomes to lysosomes for antigen digestion and presentation via the MHC class-II pathway, vaccination primarily activates CD4 T-helper cells and B cells. To combat disorder or diseases such as cancer, as well as intracellular infections, the stimulation of cytotoxic CD8 T-cell responses is important. However, the induction of cytotoxic CD8 T cells usually fails due to the difficulty in delivering antigen to the cytosol and to the MHC class-I pathway of antigen presentation. The present method allows MHC class-1 presentation of antigens on dendritic cells which therefore provides a useful vaccination method.

The use of such low doses of TPCS₂ₐ in the methods of the invention is particularly advantageous, for example, to minimise the dose of the photosensitizer to be used and hence any side effects, e.g. to minimise damage to the dendritic cell. The present invention enables the minimisation or prevention of cell death/apoptosis of the dendritic cell caused by photosensitization. This enables improved efficiency of the preparation of a dendritic cell, or populations of dendritic cells, on which an antigen is presented, for example for use in vaccination.

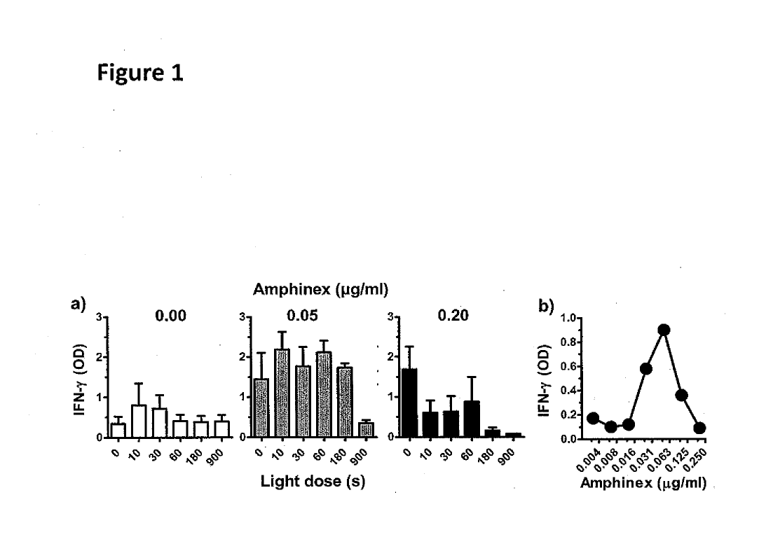

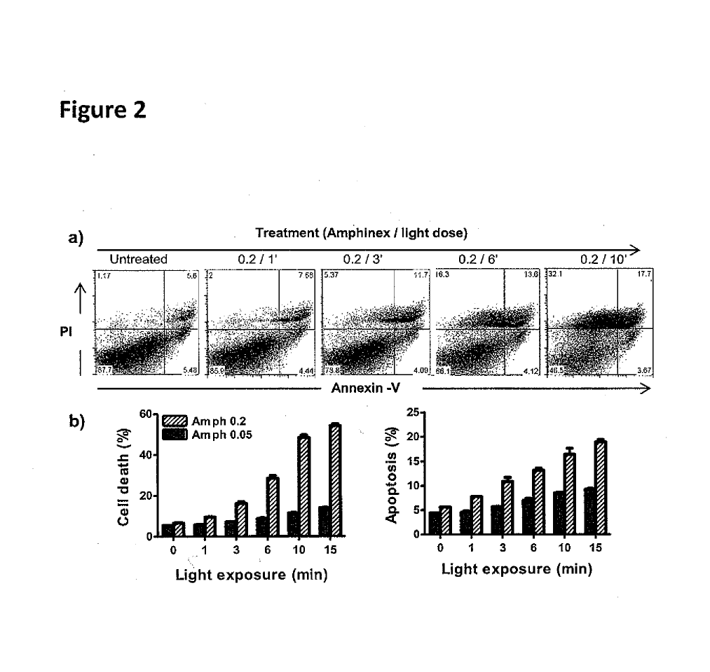

As will be described in more detail in the Examples below, it has been demonstrated that the method of the invention which employs a surprisingly low dose or amount of the photosensitizing agent TPCS₂ₐ, may be used efficiently to achieve antigen-presentation on the surface of dendritic cells. FIG. 1a and Example 1 show that a PCI method utilizing low doses such as 0.020 and 0.05 μg/ml Amphinex (TPCS₂ₐ) had a beneficial effect on the presentation of MHC-I restricted OVA antigen in vitro, FIG. 1b shows that a range of other low concentrations of Amphinex of the present invention had similar effects. FIG. 2 shows decreased dendritic cell death in the method when 0.05 μg/ml Amphinex was used, compared with 0.2 μg/ml Amphinex. Whilst 0.2 μg/ml Amphinex triggered cell death and apoptosis, 0.05 μg/ml Amphinex resulted in reduced apoptosis and cell death at all durations of exposure (FIG. 2b).

Thus, in a first aspect, the present invention provides an in vitro method of expressing an antigenic molecule or a part thereof on the surface of a dendritic cell, said method comprising:
i) contacting said dendritic cell with
   (a) an antigenic molecule, and
   (b) the photosensitising agent disulfonated tetraphenyl chlorin (TPCS₂ₐ) or a pharmaceutically acceptable salt thereof, at a concentration of 0.020-0.1 μg/ml,
wherein said antigenic molecule and said photosensitizing agent are each taken up into an intracellular vesicle; and
ii) irradiating the dendritic cell with light of a wavelength of between 400 and 500 nm, such that the membrane of the intracellular vesicle is disrupted, releasing the antigenic molecule into the cytosol of the cell,
   wherein said antigenic molecule, or a part thereof, is subsequently presented on the surface of said dendritic cell.

Preferably the photosensitizing agent is used at a range of 0.025-0.05 μg/ml, or, 0.020 (or 0.025) to less than 0.05 μg/ml.

As used herein “expressing” or “presenting” refers to the presence of the antigenic molecule or a part thereof on the surface of said dendritic cell such that at least a portion of that molecule is exposed and accessible to the environment surrounding that cell, preferably such that an immune response may be generated to the presented molecule or part thereof. Expression on the “surface” may be achieved in which the molecule to be expressed is in contact with the cell membrane and/or components which may be present or caused to be present in that membrane.

An “antigenic” molecule as referred to herein is a molecule which itself, or a part thereof, is capable of stimulating an immune response, when presented to the immune system or immune cells in an appropriate manner. Advantageously, therefore the antigenic molecule will be a vaccine antigen or vaccine component, such as a polypeptide containing entity.

Many such antigens or antigenic vaccine components are known in the art and include all manner of bacterial or viral antigens or indeed antigens or antigenic components of any pathogenic species including protozoa or higher organisms. Whilst traditionally the antigenic components of vaccines have comprised whole organisms (whether live, dead or attenuated) i.e. whole cell vaccines, in addition sub-unit vaccines, i.e. vaccines based on particular antigenic components of organisms e.g. proteins or peptides, or even carbohydrates, have been widely investigated and reported in the literature. Any such “sub-unit”-based vaccine component may be used as the antigenic molecule of the present invention. However, the invention finds particular utility in the field of peptide vaccines. Thus, a preferred antigenic molecule according to the invention is a peptide (which is defined herein to include peptides of both shorter and longer lengths i.e. peptides, oligopeptides or polypeptides, and also protein molecules or fragments thereof e.g. peptides of 5-500 e.g. 10 to 250 such as 15 to 75, or 8 to 25 amino acids).

Once released in the cell cytosol by the photochemical internalisation process, the antigenic molecule may be processed by the antigen-processing machinery of the cell and presented on the cell surface in an appropriate manner e.g. by Class I MHC. This processing may involve degradation of the antigen, e.g. degradation of a protein or polypeptide antigen into peptides, which peptides are then complexed with molecules of the MHC for presentation. Thus, the antigenic molecule expressed or presented on the surface of the cell according to the present invention may be a part or fragment of the antigenic molecule which is internalised (endocytosed). A “part” of an antigenic molecule which is presented or expressed preferably comprises a part which is generated by antigen-processing machinery within the cell. Parts may, however, be generated by other means which may be achieved through appropriate antigen design (e.g. pH sensitive bands) or through other cell processing means. Conveniently such parts are of sufficient size to generate an immune response, e.g. in the case of peptides greater than 5, e.g. greater than 10 or 20 amino acids in size.

A vast number of peptide vaccine candidates have been proposed in the literature, for example in the treatment of viral diseases and infections such as AIDS/HIV infection or influenza, canine parvovirus, bovine leukaemia virus, hepatitis, etc. (see e.g. Phanuphak et al., Asian Pac. J. Allergy. Immunol. 1997, 15(1), 41-8; Naruse, Hokkaido Igaku Zasshi 1994, 69(4), 811-20; Casal et al., J. Virol., 1995, 69(11), 7274-7; Belyakov et al., Proc. Natl. Acad. Sci. USA, 1998, 95(4), 1709-14; Naruse et al., Proc. Natl. Sci. USA, 1994 91(20), 9588-92; Kabeya et al., Vaccine 1996, 14(12), 1118-22; Itoh et al., Proc. Natl. Acad. Sci. USA, 1986, 83(23) 9174-8. Similarly bacterial peptides may be used, as indeed may peptide antigens derived from other organisms or species.

In addition to antigens derived from pathogenic organisms, peptides have also been proposed for use as vaccines against cancer or other diseases such as multiple sclerosis. For example, mutant oncogene peptides hold great promise as cancer vaccines acting as antigens in the stimulation of cytotoxic T-lymphocytes. (Schirrmacher, Journal of Cancer Research and Clinical Oncology 1995, 121, 443-451; Curtis Cancer Chemotherapy and Biological Response Modifiers, 1997, 17, 316-327). A synthetic peptide vaccine has also been evaluated for the treatment of metastatic melanoma (Rosenberg et al., Nat. Med. 1998, 4(3), 321-7). A T-cell receptor peptide vaccine for the treatment of multiple sclerosis is described in Wilson et al., J. Neuroimmunol. 1997, 76(1-2), 15-28. Any such peptide vaccine component may be used as the antigenic molecule of the invention, as indeed may any of the peptides described or proposed as peptide vaccines in the literature. The peptide may thus be synthetic or isolated or otherwise derived from an organism.

An “immune response” which may be generated may be humoral and cell-mediated immunity, for example the stimulation of antibody production, or the stimulation of cytotoxic or killer cells, which may recognise and destroy (or otherwise eliminate) cells expressing “foreign” antigens on their surface. The term “stimulating an immune response” thus includes all types of immune responses and mechanisms for stimulating them and encompasses stimulating CTLs though this is recited separately in some instances in the specification. Preferably the immune response which is stimulated is cytotoxic CD8 T cells.

The stimulation of cytotoxic cells or antibody-producing cells, requires antigens to be presented to the cell to be stimulated in a particular manner by the antigen-presenting cells, for example MHC Class I presentation (e.g. activation of CD8⁺ cytotoxic T-cells requires MHC-I antigen presentation). Preferably the immune response is stimulated via MHC-I presentation.

The method of the invention is applied to dendritic cells. Dendritic cells are immune cells forming part of the mammalian immune system. Their main function is to process antigenic material and present it on the surface to other cells of the immune system. Once activated, they migrate to the lymph nodes where they interact with T cells and B cells to initiate the adaptive immune response.

Dendritic cells are derived from hematopoietic bone marrow progenitor cells. These progenitor cells initially transform into immature dendritic cells which are characterized by high endocytic activity and low T-cell activation potential. Once they have come into contact with a presentable antigen, they become activated into mature dendritic cells and begin to migrate to the lymph node. Immature dendritic cells phagocytose pathogens and degrade their proteins into small pieces and upon maturation present those fragments at their cell surface using MHC molecules.

The dendritic cells of the invention may be derived from any appropriate source of dendritic cells, such as from the skin, inner lining of the nose, lungs, stomach and intestines or the blood. In a preferred embodiment of the present invention the dendritic cells are derived from bone marrow.

Dendritic cells may be isolated from natural sources for use in the methods of the invention or may be generated in vitro. Dendritic cells arise from monocytes, i.e. white blood cells which circulate in the body and, depending on the right signal, can differentiate into either dendritic cells or macrophages. The monocytes in turn are formed from stem cells in the bone marrow. Monocyte-derived dendritic cells can be generated in vitro from peripheral blood mononuclear cells (PBMCs). Plating of PBMCs in a tissue culture flask permits adherence of monocytes. Treatment of these monocytes with interleukin 4 (IL-4) and granulocyte-macrophage colony stimulating factor (GM-CSF) leads to differentiation to immature dendritic cells (iDCs) in about a week. Subsequent treatment with tumor necrosis factor (TNF) further differentiates the iDCs into mature dendritic cells.

The dendritic cell may be derived from any animal, including mammals, birds, reptiles, amphibians and fish. Preferably, however, the cells are mammalian, for example cells from cats, dogs, horses, donkeys, sheep, pigs, goats, cows, mice, rats, rabbits, guinea pigs, but most preferably from humans.

As used herein “contacting” refers to bringing the cells and the photosensitizing agent and and/or the antigenic molecule into physical contact with one another under conditions appropriate for internalization into the cells, e.g. preferably at 37° C. in an appropriate nutritional medium, e.g. from 25-39° C.

The cell may be contacted with the photosensitizing agent and antigenic molecule sequentially or simultaneously. Preferably, and conveniently the two components are contacted with the cell simultaneously. The contact between the cell and the photosensitizing agent and/or antigenic molecule is conveniently from 15 minutes to 12 hours, e.g. 30 minutes to four hours, preferably from 1.5 to 2.5 hours. Conveniently the cells may be placed into photosensitizer/antigen-free medium after the contact with the photosensitizer/antigen and before irradiation, e.g. for 30 minutes to 4 hours, e.g. from 1.5 to 2.5 hours.

The concentration of photosensitizing agent to be used is defined as an essential feature of the invention. The concentration of antigen to be used will depend on the antigen which is to be used. Conveniently a concentration of 5-100 μg/ml (e.g. 20-100 μg/ml or 20-50 μg/ml) antigen may be used. In the Examples, a protein with a molecular weight of 33 to 40 kDa at a concentration of 20-100 μg/ml was used. A similar molar concentration may be used for other antigens.

“Irradiation” of the cell to activate the photosensitising agent refers to the administration of light as described hereinafter. Thus cells are illuminated directly with a light source.

The light irradiation step to activate the photosensitising agent may take place according to techniques and procedures well known in the art. The wavelength of light to be used is between 400 and 500 nm, more preferably between 400 and 450 nm, e.g. from 430-440 nm, and even more preferably approximately 435 nm, or 435 nm. Suitable light sources are well known in the art, for example the LumiSource® lamp of PCI Biotech AS.

The time for which the cells are exposed to light in the methods of the present invention may vary. The efficiency of the internalisation of a molecule into the cytosol increases with increased exposure to light to a maximum beyond which cell damage and hence cell death increases.

Generally, the length of time for the irradiation step is in the order of seconds to minutes e.g. preferably from 10 seconds to 10 minutes, preferably from 10 seconds to 180 (or 300) seconds, e.g. 10-60 seconds, preferably 10-15, e.g. 15 seconds. Appropriate light doses can be selected by a person skilled in the art. For example, a light dose in the range of 0.1-6 J/cm² at a fluence range of 5-20 (e.g. 13 as provided by Lumisource®) mW/cm² is appropriate.

Pharmaceutically acceptable salts of TPCS₂ₐ are preferably acid addition salts with physiologically acceptable organic or inorganic acids. Suitable acids include, for example, hydrochloric, hydrobromic, sulphuric, phosphoric, acetic, lactic, citric, tartaric, succinic, maleic, fumaric and ascorbic acids. Hydrophobic salts may also conveniently be produced by for example precipitation. Appropriate salts include for example acetate, bromide, chloride, citrate, hydrochloride, maleate, mesylate, nitrate, phosphate, sulphate, tartrate, oleate, stearate, tosylate, calcium, meglumine, potassium and sodium salts. Amphinex as used in the Examples is a monoethanolammonium salt, and is a preferred embodiment for use in the invention. Procedures for salt formation are conventional in the art.

The photosensitizing agent and antigenic molecule may be taken up into the same or a different intracellular vesicle relative to each other. It has been found that active species produced by photosensitizers may extend beyond the vesicle in which they are contained and/or that vesicles may coalesce allowing the contents of a vesicle to be released by coalescing with a disrupted vesicle. As referred to herein “taken up” signifies that the molecule taken up is wholly containing within the vesicle. The intracellular vesicle is bounded by membranes and may be any such vesicle resulting after endocytosis, e.g. an endosome or lysosome.

As used herein, a “disrupted” vesicle or compartment refers to destruction of the integrity of the membrane of that vesicle or compartment either permanently or temporarily, sufficient to allow release of the antigenic molecule contained within it.

Preferably the method is performed without killing the cells. As used herein, the term “without killing the cell” means that a population or plurality of cells, substantially all of the cells, or a significant majority (e.g. at least 75%, more preferably at least 80, 85, 90 or 95% of the cells) are not killed. Cell viability following PCI treatment can be measured by standard techniques known in the art such as the MTS test. The methods of the current invention allow survival of a significant majority of the cells and they remain substantially functionally intact (see FIG. 2).

As cell death may not occur instantly, the % cell death refers to the percent of cells which remain viable within a few hours of irradiation (e.g. up to 4 hours after irradiation) but preferably refers to the % viable cells 4 or more hours after irradiation.

The invention further provides a dendritic cell expressing an antigenic molecule, or a part thereof, on its surface, or a population thereof, which dendritic cell is obtainable (or obtained) by a method as defined herein. Also provided is the dendritic cell or cell population for use in therapy, as described hereinafter.

The dendritic cell population may be provided in a pharmaceutical composition comprising in addition one or more pharmaceutically acceptable diluents, carriers or excipients. These compositions (and products of the invention) may be formulated in any convenient manner according to techniques and procedures known in the pharmaceutical art, e.g. using one or more pharmaceutically acceptable diluents, carriers or excipients. “Pharmaceutically acceptable” as referred to herein refers to ingredients that are compatible with other ingredients of the compositions (or products) as well as physiologically acceptable to the recipient. The nature of the composition and carriers or excipient materials, dosages etc. may be selected in routine manner according to choice and the desired route of administration, purpose of treatment etc. Dosages may likewise be determined in routine manner and may depend upon the nature of the molecule (or components of the composition or product), purpose of treatment, age of patient, mode of administration etc.

The present invention also provides a kit for use in expressing an antigenic molecule or a part thereof on the surface of a dendritic cell in a method as defined herein, said kit comprising
a first container containing a photosensitizing agent as defined herein, i.e. at a concentration of between 0.020 and 0.1 μg/ml, or a more concentrated solution of said photosensitizer for dilution to a concentration of between 0.020 and 0.1 μg/ml,
and optionally
a second container containing said antigenic molecule as defined herein.

The antigen presenting dendritic cells are prepared in vitro. In treatment methods, these cells may be administered to a body in vivo or a body tissue ex vivo such that those dendritic cells may stimulate an immune response, e.g. for therapeutic purposes.

Thus the invention further provides a dendritic cell population (or composition containing the same) as defined herein for use in stimulating an immune response or for stimulating CTLs in a subject, preferably for treating or preventing a disease, disorder or infection in said subject. Alternatively defined the present invention provides use of a dendritic cell population as defined herein for the preparation of a medicament for stimulating an immune response or for stimulating CTLs in a subject, preferably for treating or preventing a disease, disorder or infection in said subject.

In an alternative embodiment the present invention provides an antigenic molecule and a photosensitizing agent as defined herein for use in expressing said antigenic molecule or a part thereof on the surface of a dendritic cell to stimulate an immune response or for stimulating CTLs in a subject, preferably to treat or prevent a disease, disorder or infection in said subject, wherein said use comprises a method as defined herein to prepare a population of dendritic cells. The antigenic molecule and photosensitizing agent may be combined and presented in a composition. Alternatively expressed, the invention provides use of an antigenic molecule and/or a photosensitizing agent as defined herein in the manufacture of a medicament for stimulating an immune response or for stimulating CTLs in a subject, preferably to treat or prevent a disease, disorder or infection in said subject, preferably for vaccination or for treating or preventing cancer, wherein said medicament comprises a population of dendritic cells expressing an antigenic molecule or a part thereof on the surface of said dendritic cells obtainable by a method as defined herein, for administration to said subject.

Preferably the dendritic cell population is obtained by such methods. The population is for administration to the subject.

The invention further provides a product comprising an antigenic molecule and a photosensitizing agent as defined herein as a combined preparation for simultaneous, separate or sequential use in expressing said antigenic molecule or a part thereof on the surface of a dendritic cell in a method as defined herein, preferably to treat or prevent a disease, disorder or infection in a subject. The products and kits of the invention may be used to achieve cell surface presentation (or therapeutic methods) as defined herein.

In a yet further embodiment the present invention provides a method of generating an immune response or for stimulating CTLs in a subject, preferably to treat or prevent a disease, disorder or infection in said subject, comprising preparing a population of dendritic cells according to the method defined herein, and subsequently administering said dendritic cells to said subject.

The antigenic presentation achieved by the claimed invention may advantageously result in the stimulation of an immune response when the treated cells are administered in vivo. Preferably an immune response which confers protection against subsequent challenge by an entity comprising or containing said antigenic molecule or part thereof is generated, and consequently the invention finds particular utility as a method of vaccination.

The disease, disorder or infection is any disease, disorder or infection which may be treated or prevented by the generation of an immune response, e.g. by eliminating abnormal or foreign cells which may be identified on the basis of an antigen (or its level of expression) which allows discrimination (and elimination) relative to normal cells. Selection of the antigenic molecule to be used determines the disease, disorder or infection to be treated. Based on the antigenic molecules discussed above, the methods, uses, compositions, products, kits and so forth, described herein may be used to treat or prevent against, for example, infections (e.g. viral or bacterial as mentioned hereinbefore), cancers or multiple sclerosis. Prevention of such diseases, disorders or infection may constitute vaccination. As referred to herein “vaccination” is the use of an antigen (or a molecule containing an antigen) to elicit an immune response which is prophylactic against the development of a disease, disorder or infection, wherein that disease, disorder or infection is associated with abnormal expression of that antigen. In the present case the antigen is presented via treated DCs.

As referred to herein a “subject” is an animal, preferably a mammalian animal, e.g. a cow, horse, sheep, pig, goat, rabbit, cat, dog, especially preferably a human.

As defined herein “treatment” refers to reducing, alleviating or eliminating one or more symptoms of the disease, disorder or infection which is being treated, relative to the symptoms prior to treatment. “Prevention” (or prophylaxis) refers to delaying or preventing the onset of the symptoms of the disease, disorder or infection. Prevention may be absolute (such that no disease occurs) or may be effective only in some individuals or for a limited amount of time.

For in vivo administration of the cells, any mode of administration of the dendritic cell population which is common or standard in the art may be used, e.g. injection or infusion, by an appropriate route. Conveniently, the cells are administered by intralymphatic injection. Preferably 1×10⁴ to 1×10⁸ cells are administered per kg of subject (e.g. 1.4×10⁴ to 2.8×10⁶ per kg in human). Thus, for example, in a human, a dose of 0.1-20×10⁷ cells may be administered in a dose, i.e. per dose, for example as a vaccination dose. The dose can be repeated at later times if necessary.

The invention will now be described in more detail in the following non-limiting Examples with reference to the following drawings in which:

FIG. 1 shows in vitro antigen presentation with soluble OVA: (a) 100,000 bone-marrow-derived murine DCs were pulsed in 96-well plates for 2 hours with 20 μg/ml OVA and without (white histograms), or with 0.05 μg/ml (grey histograms) or 0.20 μg/ml (black histograms) of the photosensitiser Amphinex (TPCS₂ₐ). The DCs were washed and illuminated for the indicated time intervals before adding 100,000 purified CD8 T cells from OT-1 mice. IFN-gamma secretion in overnight cultures was measured by ELISA; (b) shows the same conditions as in FIG. 1a, but with various concentrations of Amphinex. After washing, the DCs were illuminated for 15 seconds.

FIG. 2 shows PCI-induced apoptosis and cell death: Bone-marrow derived murine DCs were incubated for 2 hours with the photosensitiser Amphinex at the indicated concentrations (μg/ml). The DCs were then washed and seeded into culture plates and treated with light for various time periods (minutes) as indicated. The cells were cultured for another 2 hours (or overnight) and the viability was analysed by flow cytometry after staining with Annexin-V and propidium iodide (PI). The results are illustrated as representative dotbiots (a) or summarised in histograms (b).

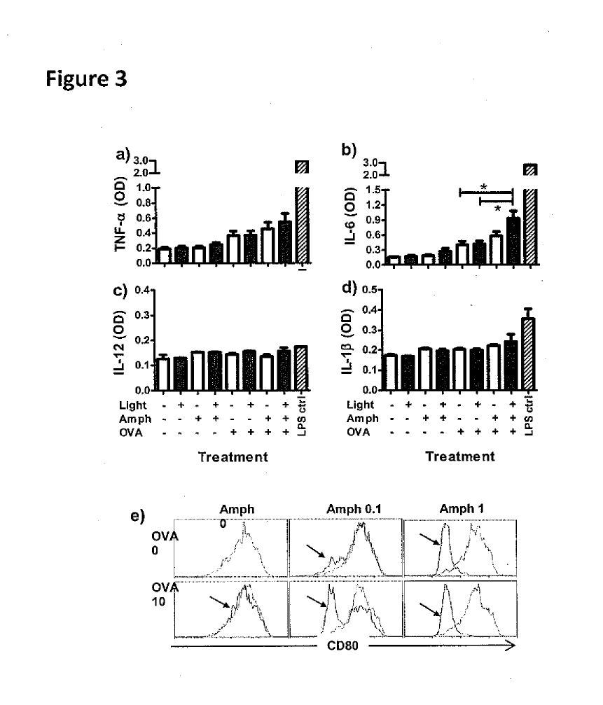

FIG. 3 shows PCI-induced activation of DCs: One million DCs were incubated with 0 or 1 μg/ml of the photosensitiser Amphinex for two hours. The cells were then washed and cultured in Amphinex- and OVA-free medium for another 2 hours before being treated with Lumisource® light for 3 minutes. The DCs were then cultured overnight before collection of supernatants for analysis of the cytokines TNF-α (a), IL-6 (b), IL-12 (c) and IL-1β (d). To measure the expression of CD80 by flow cytometry, DCs were cultured with 0, 0.1 or 1 μg/ml Amphinex and 0 or 10 μg/ml OVA as indicated (e). The histograms are representative of triplicates and show cells that were gated on viable and CD11c-positive cells. Arrows indicate samples that were treated with light for 3 min.

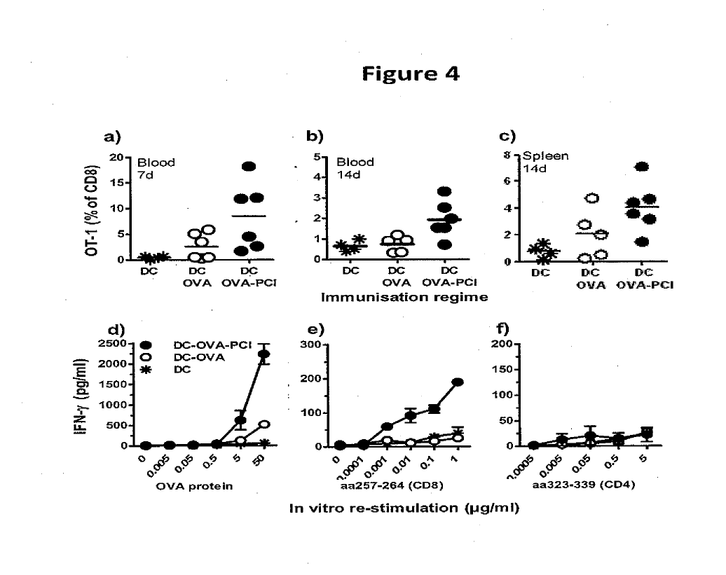

FIG. 4 shows autologous vaccination of mice with PCI-treated DCs: DCs were pulsed in vitro with 20 μg/ml OVA±0.05 μg/ml Amphinex for 2 hours; a negative control preparation comprised untreated DCs. After washing, the Amphinex-treated DCs were exposed to Lumisource® light for 3 minutes. C57BL/6 mice were immunised with 2×10⁶ DCs of either DC preparation by intralymphatic injection (inguinal LN). Prior to immunisation, the mice received 10⁷ purified OT-1 CD8 T cells (i.p.). Mice were bled on days 7 (a) and 14 (b) for analysis of OT-1-specific cells by flow cytometry. On day 14, the frequency of OT-1-specific cells were also analysed in splenocytes (c). The splenocytes were also re-stimulated in vitro with OVA protein (d) or peptides (e-f) for determination of antigen-specific secretion of IFN-gamma in supernatants by ELISA.

### EXAMPLES

### Example 1

### Preparation of Antigen-Presenting Dendritic Cells and Administration to Mice to Generate an Immune Response

### Materials and Methods

### Mice

For immunisation as well as for preparation of bone-marrow dendritic cells (DCs), C57BL16 mice were purchased from Harlan (Horst, The Netherlands). OT-I mice transgenic for the T-cell receptor that recognises the MHC class-I restricted epitope OVA₂₅₇₋₂₆₄ from ovalbumin (OVA) were bred in facilities at the University of Zurich. All mice were kept under specified pathogen-free (SPF) conditions, and the procedures performed were approved by Swiss Veterinary authorities.

### Bone-Marrow Derived Dendritic Cells (DCs)

Mouse DCs were prepared by isolating bone marrow cells from femurs. Briefly, femurs were aseptically harvested and bone marrow cells cultured in DMEM medium (Brunschwig, Basel, Switzerland) supplemented with 10% FCS, glutamine, sodium pyruvate, penicillin and streptomycin in the presence of 10% supernatant from GM-CSF-secreting X-63 cells; the X-63 cell line was transfected and kindly provided by Dr. A. Rolink (University of Basel). After six to seven days, the loosely adherent dendritic cells (DCs) were harvested by flushing, with medium and the collected DCs were washed once and re-suspended in fresh medium for further use.

### Isolation of OT-1 CD8 Positive T Cells

Spleens and lymph nodes were isolated from OT-1 mice, and erythrocytes were removed by lysis (RBC Lysing Buffer Hybri-Max from Sigma-Aldrich). CD8-positive T cells were then purified using magnetic anti-mouse CD8a (Ly-2) MicroBeads as described by the provider (Miltenyi Biotech, Bergisch Gladbach, Germany).

### In Vitro Studies of Antigen Presentation and Photochemical Internalisation

The antigen OVA (Sigma Aldrich, Buchs, Switzerland) and the photosensitiser Amphinex (TPCS₂ₐ, PCI Biotech, Lysaker, Norway) were incubated with DCs using petri dishes. Typically, DCs were pulsed for 2 h with OVA and Amphinex. The DCs were then collected and washed by centrifugation before re-suspension in medium and further incubation on petri dishes for 2 h in Amphinex- and OVA-free medium; this allows removal of Amphinex from the outer plasma membrane. The DCs were then washed, counted and plated in round-bottom 96-well plates (typically 100,000 DCs per well), and the cells were exposed to light (435 nm) using LumiSource® (PCI Biotech) for different time intervals. Sex-matched CD8-purified OT-1 cells were then added to the DC plates at 100,000 cells per well and incubated at 37° C. overnight. The secretion of IFN-γ into supernatants was measured using ELISA according to the protocol from eBioscience (Ready-SET-Go!®, Bender MedSystems, Vienna, Austria).

### Activation, Apoptosis and Viability Testing of DCs In Vitro

The DC viability was tested 2 hours after the PCI treatment by staining the cells with propidium iodide and Annexin-V to identify necrotic and apoptotic cells, respectively, which were analysed by flow cytometry (FACSCanto from BD Biosciences, San Jose, USA). The analysis was performed using the FlowJo 8.5.2 software from Tree Star, Inc. (Ashland, Oreg.). Activation of DCs was further tested by measuring the secretion of TNF-α, IL-6, IL-12 and IL-1β by ELISA (eBioscience) and the expression of MHC I, MHC II, CD40, CD80, CD83 and CD86 by flow cytometry. Briefly, the DCs were incubated for 2 hours with Amphinex or lipopolysaccharide E. coli clone 026:B6 (Sigma Aldrich), washed, incubated for another 2 hours in fresh medium, illuminated for 3 min with Lumisource® light, and incubated. Cytokines were analysed by ELISA from supernatants of 24 hours cultures, and flow cytometry was done on cells after 48 hours incubation. All FACS antibodies were purchased from BD Pharmingen (Basel, Switzerland) or from eBioscience.

Autologous Immunisation of Mice with PCI-Treated DCs

The feasibility of applying PCI to vaccination was tested in mice by intralymphatic injection of antigen-pulsed and PCI-treated DCs. The DCs were loaded with soluble OVA (20 μg/ml) and Amphinex (0.05 μg/ml) as described above, washed and exposed to light for 3 minutes. The numbers of DCs injected into one inguinal lymph node in C57BL/6 mice was 2×10⁶. One day prior to the immunisation, the mice received 10⁷ OT-1 cells by intraperitoneal injection; the adoptive transfer OVA₂₅₇₋₂₆₄-specific CD8 T cells allows better monitoring of the immune response by flow cytometry. On day 7 and 14 the OVA-specific CD8 T cells in blood was monitored by staining mouse PBMC with anti-CD8 antibody and H-2K^{b}/OVA₂₅₇₋₂₆₄ Pro5 pentamer (Proimmune, Oxford, UK) for analysis of the frequency of OVA-specific T-cells in vivo by flow cytometry. On day 14, the mice were euthanized and the number of OVA-specific T cells in spleens determined by anti-CD8 and pentamer staining. Splenocytes were re-stimulated with OVA protein, CD8 epitope OVA₂₅₇₋₂₆₄ or the CD4 epitope OVA₃₂₃₋₂₃₈ for analysis of OVA specific CD4 and CD8 T cell activation. After 72 hours, cell supernatants were collected and the content of IFN-γ was determined by ELISA.

### Results

### PCI Increased Antigen Presentation of Protein In Vitro

To test the effect of photosensitiser Amphinex and light on the enhancement of MHC class-I-restricted antigen presentation, mouse bone-marrow DCs were grown in 10 cm petri dishes and pulsed with OVA for 2 hours without or with 0.05 or 0.2 μg/ml Amphinex. After removing the photosensitiser and antigen by washing and incubation for another 2 hours, the DCs were transferred to 96-well plates at 100,000 cells per well and treated with light at different time-doses before admixing an equal number of purified OT-1 CD8 T cells. PCI had a beneficial effect on the presentation of MHC-1 restricted OVA antigen in vitro (FIG. 1a). While OVA-treated DCs show some degree of MHC-1 antigen presentation as measured by IFN-γ secretion after 24 hours, presentation was significantly increased when the DCs were treated with Amphinex. A combination of Amphinex and high light doses had a clear detrimental effect on the antigen presentation. This latter effect and the fact that stronger immune responses were typically observed with 0.05 μg/ml than with 0.2 μg/ml Amphinex, may suggest residuals of Amphinex in the outer cell membrane of DCs which then become sensitive to environmental light. Of note, antigen presentation and IFN-γ secretion of soluble OVA was improved by Amphinex alone, but not by light alone (FIG. 1a). To further test the sensitivity of DCs to PCI treatment, equivalent assays were performed using a single light dose of 15 seconds, but a wide range of Amphinex doses (0.0005-0.5 μg/ml), namely 0.003, 0.006, 0.0125, 0.025, 0.05, 0.1 and 0.2 μg/ml. A representative test is shown in FIG. 1b. The illumination of DCs resulted in Amphinex-dose-dependent IFN-γ secretion by OT-1 CD8 T cells. Amphinex concentrations of 0.025 and 0.05 μg/ml facilitated antigen presentation, whereas the IFN-γ secretion declined at higher Amphinex doses.

### PCI Induces Apoptosis in Bone-Marrow-Derived DCs

Initial experiments suggested that the viability and capability of presenting antigen was strongly compromised by the application of light to cultures of Amphinex-treated DCs. Therefore in a series of experiments, we analysed cell death and apoptosis by flow cytometry after staining of cells with propidium iodide and fluorescence-labelled anti-Annexin-V. A concentration of 0.2 μg/ml Amphinex induced light-dose dependent apoptosis and cell death upon illumination with 435 nm light for 1 to 10 minutes (FIG. 2a); the DCs were rested at 37° C. for 2 hours after light treatment before staining and acquisition. The amount of cells dying under these conditions was approx. 50-60% and approx. 20% were apoptotic (FIG. 2a). The susceptibility to enter apoptosis and cell death under these experimental conditions was also Amphinex-dose dependent. While 0.2 μg/ml Amphinex triggered cell death and apoptosis, 0.05 μg/ml Amphinex resulted in reduced apoptosis and cell death at all lengths of exposure (FIG. 2b).

We further tested the activation of DCs and their innate immune reactions after PCI treatment. Adjuvants and especially pathogen-associated molecular patterns (PAMPs) typically activate DCs and other antigen presenting cells via stimulation of pathogen recognition receptors such as Toll-like receptors, NOD-like receptors, C-type lectin and mannose receptors. Such activation then is typically characterised by secretion cytokines important for stimulation and regulation of further innate as well as adoptive immune responses. PCI treatment caused only weak stimulation of TNF-α (FIG. 3a) and IL-6 (FIG. 3a) secretion when measured after 22 hours incubation of DC cultures. Although weak, the adjuvant effect with regard to IL-6 seemed light dependent, as IL-6 secretion was not increased by Amphinex or light alone, but only by their combination. However, the effect was much weaker than after stimulation of DCs with 1 μg/ml lipopolysaccharide (LPS). While stimulation of TNF-α and IL-6 secretion characterises the general adjuvant potential of a compound or a treatment, stimulation of IL-12 and IL-1β illustrates the potential to trigger Th1 T-cell responses and inflammasome, respectively. We therefore analysed the secretion of these two cytokines and found that neither were notably stimulated by PCI treatment of DCs (FIG. 3c-d).

DC constitutively expressed the co-stimulatory molecules CD80 and CD86 (FIG. 3e; CD80 only shown). Both molecules were rapidly up-regulated after stimulation with 1 μg/ml LPS (not shown), but not after stimulation with Amphinex or OVA. However Amphinex-treated DCs that were also treated with light showed a down-regulation of CD80, independent of being pulsed with OVA or not (FIG. 3e); the DCs were gated on viable and CD11c-positive cells; hence, the down-regulation was not a result of cell death. The expression of CD40, MHC I and MHC II was not affected by Amphinex treatment (not shown).

Autologous Immunisation with PCI-Treated DCs Trigger Antigen-Specific T-Cell Proliferation and Cytokine Secretion

To analyse whether PCI-treated DCs promoted the stimulation of antigen-specific CD8 T-cell responses in vivo, mice were immunised by intralymphatic injection of 2 million antigen-pulsed DCs. The DCs were prepared in vitro with 20 μg/ml OVA, 0.05 μg/ml Amphinex and 3 minutes light at 435 nm as described above. One day prior to immunisation, the mice were spiked by adoptive transfer of splenocytes from OT-1 mice to facilitate detection of antigen-specific CD8 T cells by flow cytometry. PCI-treatment of the DCs increased the stimulation of antigen-specific T-cell proliferation as monitored by the frequency of OT-1 specific cells CDB T cells in blood 7 and 14 days after immunisation compared to immunisation with OVA-loaded DCs that were not PCI-treated. The means of specific cells out of the whole CD8 populations were 8.4% for DC-OVA-PCI, 2.6% for DC-OVA, and 0.44% for sham-treated (DC alone) mice on day 7 (FIG. 4a). By day 14, the frequencies of antigen-specific cells in blood (FIG. 4b) and spleen (FIG. 4c) decreased strongly as expected due to the retraction of effector cells. However, mice immunised with PCI-treated DCs still showed higher frequencies of antigen-specific CD8 T cells than did control mice that received OVA-pulsed DCs that had not been PCI treated.

When splenocytes were re-stimulated in vitro with OVA for 3 days, we observed stronger secretion of IFN-γ by cells from mice immunised with PCI-treated DCs (FIG. 4d). The amount of cytokine secreted was higher in the DC-OVA-PCI group and the onset of cytokine secretion was observed at concentrations only one tenth of that required in splenocytes from mice immunised with DC-OVA without PCI (0.5 versus 5.0 μg/ml OVA). The IFN-γ secretion was not a polyclonal effect, but OVA₂₅₇₋₂₆₄ dependent as demonstrated in experiments with splenocytes re-stimulated with the short CD8 T-cell epitope (FIG. 4e). Splenocytes from mice immunised with DC-OVA-PCI showed reactivation for IFN-γ secretion at 0.001 μg/ml peptide, whereas splenocytes from mice treated with DC-OVA showed no OVA₂₅₇₋₂₆₄-specific IFN-γ secretion, even at 1000-fold higher peptide concentrations. No IFN-γ secretion was observed in splenocyte cultures re-stimulated with the CD4 epitope OVA₃₂₃₋₃₃₉ (FIG. 4f).

### Discussion

These results show that PCI triggered presentation of CD8 epitopes via the MHC class I pathway of antigen presentation in DCs in vitro, and autologous vaccination with DCs that had been treated in vitro with PCI caused improved antigen-specific proliferation and cytokine secretion in mice. It is evident from the use of a long protein OVA protein as the antigen, and not just merely the short MHC-class-binding epitope OVA₂₅₇₋₂₆₄, that proliferation and cytokine secretion must stem from antigen uptake, digestion in proteasomes, and MHC class-I antigen presentation to the OVA₂₅₇₋₂₆₄ reactive transgenic CD8 T cell used as the target in this study.

1. An in vitro method of expressing an antigenic molecule or a part thereof on the surface of a dendritic cell, said method comprising:
i) contacting said dendritic cell with
(a) an antigenic molecule, and
(b) the photosensitising agent disulfonated tetraphenyl chlorin (TPCS₂ₐ) or a pharmaceutically acceptable salt thereof, at a concentration of 0.020-0.1 μg/ml,
wherein said antigenic molecule and said photosensitizing agent are each taken up into an intracellular vesicle; and
ii) irradiating the dendritic cell with light of a wavelength of between 400 and 500 nm, such that the membrane of the intracellular vesicle is disrupted, releasing the antigenic molecule into the cytosol of the cell,
wherein said antigenic molecule, or a part thereof, is subsequently presented on the surface of said dendritic cell.

2. The method as claimed in claim 1 wherein the concentration of the photosensitising agent is 0.020-0.05 μg/ml.

3. The method as claimed in claim 1 or claim 2 wherein the concentration of the photosensitising agent is 0.05 μg.

4. The method as claimed in any one of claims 1-3 wherein the light has a wavelength of 430-440 nm, preferably 435 nm.

5. The method as claimed in any one of claims 1-4 wherein the dendritic cell is a bone marrow-derived dendritic cell.

6. The method as claimed in any one of claims 1-5 wherein the antigenic molecule is a molecule capable of stimulating an immune response.

7. The method as claimed in claim 6 wherein the antigenic molecule is a vaccine antigen or vaccine component.

8. The method as claimed in any one of claims 1-7 wherein the antigenic molecule is a peptide.

9. The method as claimed in any one of claims 1-8 wherein said irradiation is conducted for 10-180 seconds.

10. The method as claimed in any one of claims 1-9 wherein the antigenic presentation results in the stimulation of an immune response.

11. A dendritic cell expressing an antigenic molecule, or a part thereof, on its surface, or a population thereof, which dendritic cell is obtainable by a method as defined in any one of claims 1 to 10.

12. A pharmaceutical composition comprising a cell population as defined in claim 11 and one or more pharmaceutically acceptable diluents, carriers or excipients.

13. A dendritic cell or cell population as defined in claim 11 or a composition as defined in claim 12 for use in therapy.

14. A dendritic cell or cell population as defined in claim 11 or a composition as defined in claim 12 for use in stimulating an immune response or for stimulating CTLs in a subject, preferably for treating or preventing a disease, disorder or infection in said subject, preferably for vaccination.

15. A dendritic cell, cell population or composition for use as claimed in claim 14, for treating or preventing cancer.

16. Use of a cell population as defined in claim 11 for the preparation of a medicament for stimulating an immune response or for stimulating CTLs in a subject, preferably for treating or preventing a disease, disorder or infection in said subject, preferably for vaccination or for treating or preventing cancer.

17. A use as claimed in claim 16, wherein said stimulation, treatment or prevention comprises administering said medicament to said subject.

18. An antigenic molecule and a photosensitizing agent as defined in any one of claims 1-3 and 6-8 for use in expressing said antigenic molecule or a part thereof on the surface of a dendritic cell to stimulate an immune response or for stimulating CTLs in a subject, preferably to treat or prevent a disease, disorder or infection in said subject, preferably for vaccination or for treating or preventing cancer, wherein said use comprises a method as defined in any one of claims 1 to 10 to prepare a population of dendritic cells.

19. The antigenic molecule and photosensitizing agent for use as claimed in claim 18, wherein said population of dendritic cells are to be administered to said subject.

20. Use of an antigenic molecule and/or a photosensitizing agent as defined in any one of claims 1-3 and 6-8 in the manufacture of a medicament for stimulating an immune response or for stimulating CTLs in a subject, preferably to treat or prevent a disease, disorder or infection in said subject, preferably for vaccination or for treating or preventing cancer, wherein said medicament comprises a population of dendritic cells expressing an antigenic molecule or a part thereof on the surface of said dendritic cells obtainable by a method as defined in any one of claims 1 to 10, for administration to said subject.

21. A use as claimed in claim 20 wherein said antigenic molecule and/or photosensitizing agent are used in a method as defined in any one of claims 1 to 10 to obtain said population of dendritic cells for manufacture of said medicament.

22. A product comprising an antigenic molecule and a photosensitizing agent as defined in any one of claims 1-3 and 6-8 as a combined preparation for simultaneous, separate or sequential use in expressing said antigenic molecule or a part thereof on the surface of a dendritic cell in a method according to any one of claims 1-10, preferably to treat or prevent a disease, disorder or infection in a subject.

23. A kit for use in expressing an antigenic molecule or a part thereof on the surface of a dendritic cell in a method according to any one of claims 1-10, said kit comprising
a first container containing a photosensitizing agent as defined in any one of claims 1-3; and optionally
a second container containing said antigenic molecule as defined in any one of claims 1 and 6-8.

24. A method of generating an immune response or for stimulating CTLs in a subject, preferably to treat or prevent a disease, disorder or infection in said subject, preferably for vaccination or for treating or preventing cancer, comprising preparing a population of dendritic cells according to the method of any one of claims 1-10, and subsequently administering said dendritic cells to said subject.
